(19) United States
(12) Patent Application Publication
(10) Pub. No.: US 2016/0007981 A1
(43) Pub. Date: Jan. 14, 2016

(54) A RADIALLY-SHAPED RETRACTOR AND ASSOCIATED METHOD OF USE

(71) Applicants: Sudhanva Govindarajan, Akron, OH (US); Ashley Wilkinson, Massillon, OH (US); Orit Itzhak-Yasu, Miami Beach, FL (US); Eric Espinal MD, Akron, OH (US)

(72) Inventors: Sudhanva Govindarajan, Akron, OH (US); Ashley Wilkinson, Massillon, OH (US); Orit Itzhak-Yasu, Miami Beach, FL (US); Eric Espinal MD, Akron, OH (US)

(21) Appl. No.: 14/769,729

(22) PCT Filed: Feb. 24, 2014
(86) PCT No.: PCT/US14/17952

§ 371 Date: Aug. 21, 2015

Related U.S. Application Data
(60) Provisional application No. 61/768,034, filed on Feb. 22, 2013.

(51) Int. Cl. A61B 17/02; A61B 19/00

(52) U.S. Cl. CPC A61B 17/02; A61B 19/5202; A61B 2017/0237; A61B 2017/0287; A61B 2017/00867

(57) ABSTRACT

A method of using a retractor for holding open an opening includes the steps of providing a retractor, wherein the retractor includes a shape memory support including a brace portion connecting opposed sides and a plurality of bracing ribs extending from the shape memory support at the opposed sides, forming a surgical opening, pinching the opposed sides of the shape memory support in an inward direction, inserting the pinched retractor of said step of pinching into the surgical opening, and thereafter allowing the opposed sides of the shape memory position to substantially revert to their original positions.

### CROSS-REFERENCE TO RELATED APPLICATIONS

This application claims priority from U.S. Provisional Application No. 61/768,034 filed on Feb. 22, 2013, the contents of which are incorporated herein by reference.

### FIELD OF THE INVENTION

The present invention relates to a medical device and an associated method of use. The present invention further relates to a self-supported, radially-shaped retractor that can be used during surgical procedures, particularly surgical procedures relating to the left atrium of a human heart.

### BACKGROUND OF THE INVENTION

In some surgeries related to the heart, particularly those related to mitral valve replacement, retractors are utilized to keep an atrium, particularly the left atrium, open for the surgery to occur. However, commercially available atrial retractors fail to fully support the incision, and tend to open the incision in only one direction. These retractors can even lead to folding or creasing of the valve orifice that is being operated on, causing the need for time consuming and tedious readjustment. Further, these retractors are usually attached to a sternal retractor by way of an arm, which leaves little control for the surgeon, or are physically held by a surgical assistant which prevents the assistant from performing other surgical duties.

One such atrial retractor is a pronged inverted tool that hinges off a sternal retractor. The prongs of this retractor hold open the opening, such as a left atrium. This retractor has the shape of a curved upside-down trident and is generally affixed to a chest retractor that holds open a sternum. The angle of this retractor does not expose the surgical location well and provides a poor line of sight. The retractor usually requires readjustment throughout the surgery, wasting precious operating time.

These constraints exhibited by known retractors limit both the vision and the maneuverability of a surgeon during an operation. For many ‘complex’ hearts, the tunneling and angle of the left atrium during mitral valve surgery renders visibility scarce.

Thus, it is desired to provide an improved anatomical retractor. It is further desired to provide an improved retractor to be utilized during a surgery, particularly a heart surgery. The present invention provides one or more of the following improvements over the known art: improved support of the opening, better maneuverability for an operating surgeon, easier access to the location of a surgery, easier placement and removal, compatible with patient anatomy, and improved luminosity at the surgery location.

### SUMMARY OF THE INVENTION

In a first embodiment, the present invention provides a retractor for medical procedures comprising a shape memory support including a brace portion connecting opposed sides and a plurality of bracing ribs extending from the shape memory support at the opposed sides.

In a second embodiment, the present invention provides a retractor as in the first embodiment, wherein the periphery of the shape memory support formed by the brace portion and the opposed sides extends through at least π/2 radians.

In a third embodiment, the present invention provides a retractor as in either the first or second embodiments, wherein the periphery of the shape memory support formed by the brace portion and the opposed sides extends through from π/2 radians to 16π/9 radians.

In a fourth embodiment, the present invention provides a retractor as in any of the first through third embodiments, wherein the shape memory support is formed of a composition selected from the group consisting of spring steel, polycarbonate, and polyurethane.

In a fifth embodiment, the present invention provides a retractor as in any of the first through fourth embodiments, wherein the shape memory support is coated with a coating selected from the group consisting of silicone, polybutadiene, rubber, and a carbon composite.

In a sixth embodiment, the present invention provides a retractor as in any of the first through fifth embodiments, wherein the bracing ribs are formed of a composition selected from the group consisting of spring steel, polycarbonate, and polyurethane.

In a seventh embodiment, the present invention provides a retractor as in any of the first through sixth embodiments, wherein the bracing ribs are coated with a coating selected from the group consisting of silicone, polybutadiene, rubber, and a carbon composite.

In an eighth embodiment, the present invention provides a retractor as in any of the first through seventh embodiments, wherein the plurality of bracing ribs are substantially parallel.

In a ninth embodiment, the present invention provides a retractor as in any of the first through eighth embodiments, wherein the shape memory support has a length of from 100 mm to 200 mm.

In a tenth embodiment, the present invention provides a retractor as in any of the first through ninth embodiments, wherein the shape memory support forms a semi-elliptical shape having an overall major axis of from 3 mm to 7 mm and an overall minor axis of from 2.5 mm to 6 mm.

In an eleventh embodiment, the present invention provides a retractor as in any of the first through tenth embodiments, wherein the bracing ribs have a length of from 20 mm to 45 mm.

In a twelfth embodiment, the present invention provides a retractor as in any of the first through eleventh embodiments, wherein the opposed sides each provide a distal end having a tab extending therefrom.

In a thirteenth embodiment, the present invention provides a retractor as in any of the first through twelfth embodiments, wherein the bracing ribs include an aperture for inserting a suture.

In a fourteenth embodiment, the present invention provides a retractor as in any of the first through thirteenth embodiments, wherein the aperture is located near an end of the bracing ribs.

In a fifteenth embodiment, the present invention provides a retractor as in any of the first through fourteenth embodiments, wherein the retractor further comprises a light source.

In a sixteenth embodiment, the present invention provides a method of using a retractor for holding open an opening comprising the steps of providing a retractor, wherein the retractor comprises a shape memory support including a brace portion connecting opposed sides and a plurality of bracing ribs extending from the shape memory support at the opposed sides, forming a surgical opening, pinching the opposed sides of the shape memory support in an inward direction, inserting the pinched retractor of said step of pinching into the surgical opening, and thereafter allowing the opposed sides of the shape memory position to substantially revert to their original positions.

In a seventeenth embodiment, the present invention provides a method as in the sixteenth embodiment, wherein the periphery of the shape memory support formed by the brace portion and the opposed sides extends through at least π/2 radians.

In an eighteenth embodiment, the present invention provides a method as in either the sixteenth or seventeenth embodiments, wherein the retractor is inserted into a left atrium.

In a nineteenth embodiment, the present invention provides a method as in any of the sixteenth through eighteenth embodiments, wherein the opposed sides each provide a distal end having a tab extending therefrom, and the method further comprises the step of engaging the tabs with a medical tool in order to pinch the opposed sides of the shape memory support in an inward direction.

In a twentieth embodiment, the present invention provides a method as in any of the sixteenth through nineteenth embodiments, wherein the bracing ribs include an aperture for inserting a suture, and the method further comprises the step of suturing the retractor in a desired location.

### BRIEF DESCRIPTION OF THE DRAWINGS

FIG. 1 is a perspective view of an embodiment of the present invention.

FIG. 2 is a schematic view of a representation of a human heart that shows the location of the mitral valve.

FIG. 3 is a top view of an embodiment of the present invention.

FIG. 4 is a side view of an embodiment of the present invention.

FIG. 5 is a front view of an embodiment of the present invention.

FIG. 6 is a perspective view of an embodiment of the present invention.

FIG. 7 is a top view of an embodiment of the present invention.

FIG. 8 is a schematic view representative of a step in a method of use.

FIG. 9 is a schematic view representative of a step in a method of use.

FIG. 10 is a top view of an embodiment of the present invention.

FIG. 11 is a schematic view showing a shape memory support having a radial angle θ.

FIG. 12 is a schematic view showing a shape memory support having a radial angle θ.

FIG. 13 is a schematic view showing a shape memory support having a radial angle θ.

### DETAILED DESCRIPTION OF ILLUSTRATIVE EMBODIMENTS

The present invention relates to a medical device and an associated method of use. In one or more embodiments, the present invention further relates to a self-supported, radially-shaped retractor that can be used during surgical procedures, particularly surgical procedures relating to the left atrium of a human heart.

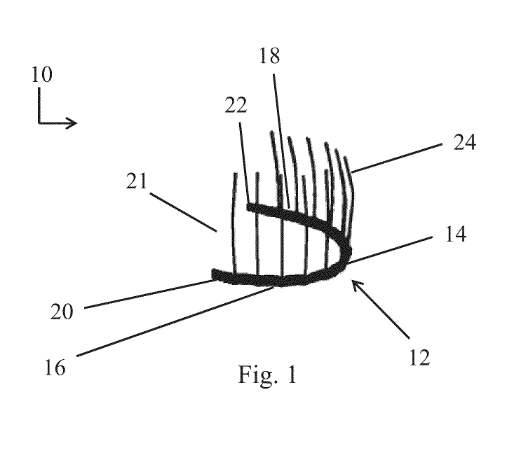

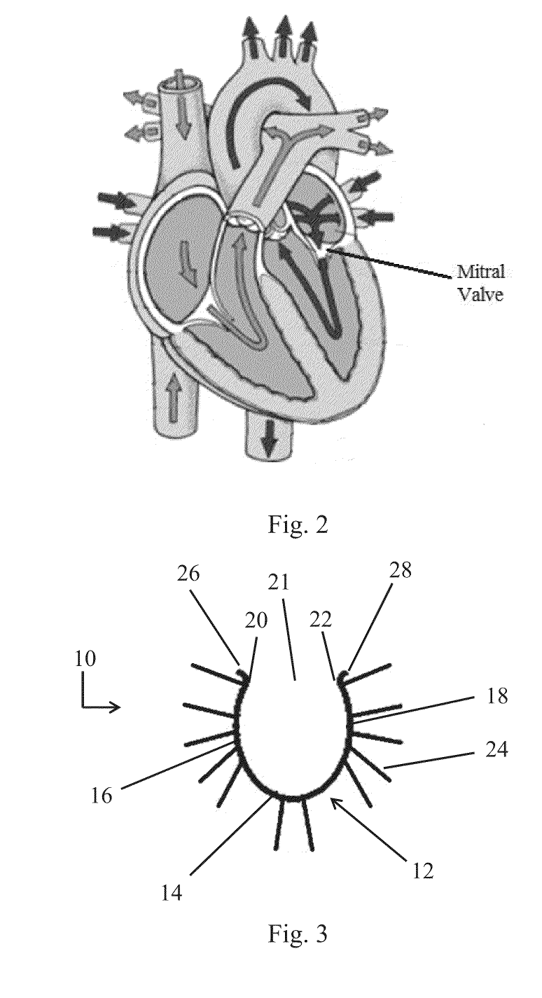

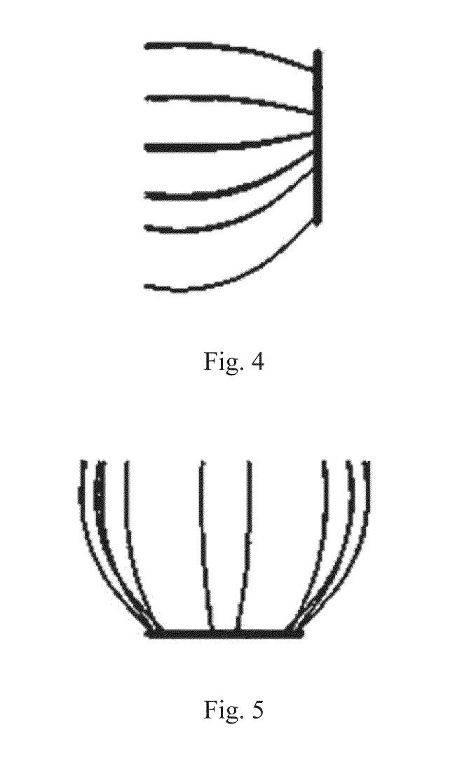

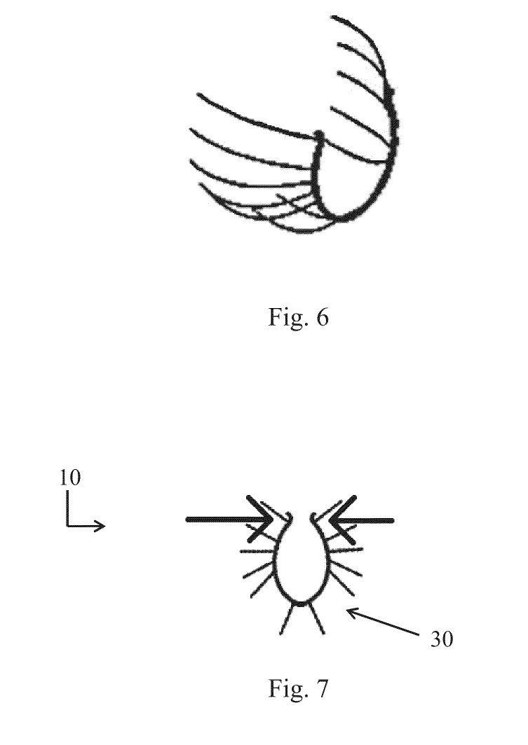

With reference to FIGS. 1 and 3, one embodiment of the present invention provides a retractor 10 comprising a shape memory support 12 and a plurality of bracing ribs 24. The retractor 10 is designed to be self-supporting in opening an incision. As described herein, self-supporting means that a retractor can keep an incision open by use of the retractor alone, once the retractor is placed inside the opening.

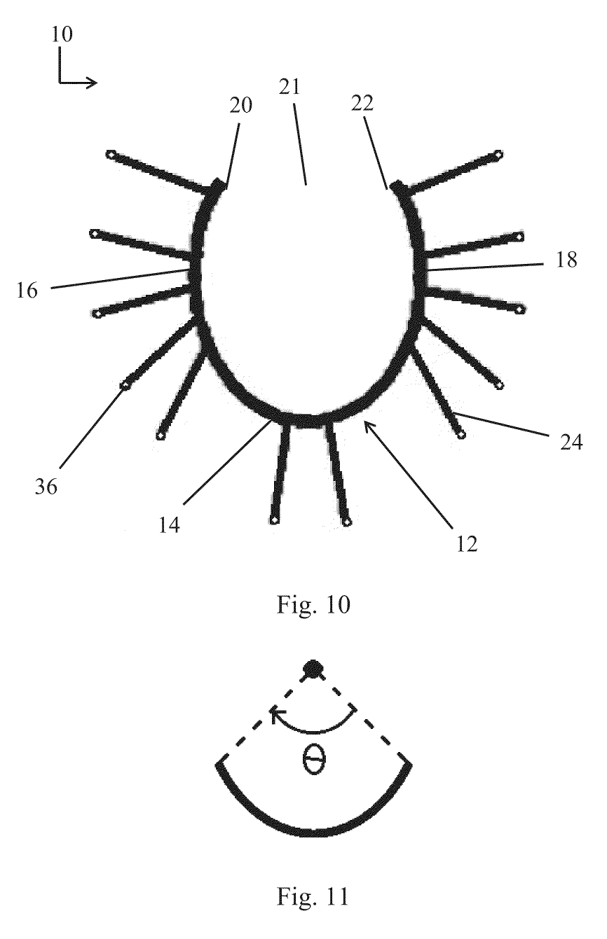

The shape memory support 12 includes a brace 14 that joins opposed sides 16, 18. The brace 14 and opposed sides 16, 18 are integral and curvilinear. The brace 14, side 16, and side 18 can each contain one or more bracing ribs 24. The side 16 provides a distal end 20 and side 18 provides distal end 22. As shown in FIGS. 1 and 10, the distal ends 20, 22 can themselves serve as means for manipulating the retractor 10. As shown in FIG. 3, distal ends 20, 22 can include tabs 26, 28 that can serve as means for manipulating the retractor. It should also be appreciated that the retractor 10 can be manipulated by a person or a tool pinching together the opposing sides 16, 18.

Distal ends 20, 22, sides 16, 18, or tabs 26, 28 when present, can be used as means for manipulation to compress shape memory support 12, bringing the opposed sides closer together. In one or more embodiments, the means for manipulation can be grasped by an individual or by a medical tool in order to compress shape memory support 12. By nature of its shape memory, releasing the compressive force results in decompression, the shape memory support 12 reverting toward its original shape. In one or more embodiments, the means for manipulation can be sized such that an individual's fingers or thumb can be used to compress shape memory support 12. The means for manipulation and the retractor 10 are designed for easy entry into, and removal from, an opening, such as a surgical incision. Also, bracing ribs 24 are of a shape and length suitable for bracing the inner walls or edges of an opening and/or inner walls to which the incision provides access. For example, an incision to the left atrium of a heart will provide access to the inner walls of the left atrium and the bracing ribs 24 can engage those inner walls of the left atrium.

The term shape memory, as used herein, is now further defined. Shape memory support 12 is designed of a flexible material such that, when shape memory support 12 is manipulated, such as being pinched or squeezed in an inner direction, the overall shape formed by shape memory support compresses, with the opposed sides coming closer together. The brace 14 will be compressed to have a shorter diameter. This is particularly advantageous for compressing shape memory support 12 to a shape that permits it to be inserted (or installed) in an incision. Once compressed, retractor 10 can be inserted into the incision, and after the compressive force is released, retractor 10 decompresses toward its rest shape, or original diameter, this decompression causes the retractor 10 to apply force to the inner walls or edges of the incision and/or inner walls to which the incision provides access. Thus, retractor 10 provides support for an opening or incision, while holding itself in place. Once inserted into the opening, the bracing ribs 24 support the retractor 10 and hold open the opening. The bracing ribs 24 in some uses will engage the inner walls to which the incision provides access. In a particular application of the retractor 10, the bracing ribs 24 will engage the inner walls of the left atrium.

The retractor 10 includes an open end 21 and a closed end. The closed end is generally defined by the arc formed by the brace 14 of shape memory support. The open end is generally defined by the opening that remains in the arc shape, defined between the opposed distal ends 20, 22 of the opposed sides 16, 18.

In one or more embodiments, the shape memory support 12 is generally curvilinear, being a semi-oval or semi-circular or semi-elliptical at the brace and/or the brace and opposed sides. In some embodiments, the opposed sides may be straight, with the curvilinear shape being provided by the brace 14. In other embodiments, such as that shown, the opposed side 16, 18 are also curvilinear in addition to the brace 14. The curvilinear shape can serve to provide comprehensive support when the retractor 10 is placed inside an incision opening. As shown in FIG. 3, the shape memory support 12 can have a shape similar to that of a horseshoe, where the shape memory support 12 includes tabs 26, 28. In one or more embodiments, the shape memory support 12 is designed in such a shape to match the shape of a location desired for a surgical procedure, such as for keeping the left atrium open during a mitral valve replacement.

In one or more embodiments, a semi-ellipse formed by the retractor 10 has an overall major axis of 5 mm, or approximate thereto, and an overall minor axis of 4.5 mm, or approximate thereto. In one or more embodiments, a semi-ellipse formed by the retractor 10 has an overall major axis of from 3 mm or more to 7 mm or less and an overall minor axis of from 2.5 mm or more to 6 mm or less. In one or more embodiments, a semi-ellipse formed by the retractor 10 has an overall major axis of from 1 mm or more to 50 mm or less and an overall minor axis of from 1 mm or more to 50 mm or less.

In one or more embodiments, the shape memory support 12 has an overall length of 160 mm. In one or more embodiments, the shape memory support 12 has an overall length of from 100 mm or more to 200 mm or less. In one or more embodiments, the shape memory support 12 has an overall length of from 20 mm to 500 mm.

In one or more embodiments, the shape memory support 12 has a thickness of 0.8 mm. In one or more embodiments, the shape memory support 12 has a thickness of from 0.5 mm or more to 1 mm or less. In one or more embodiments, the shape memory support 12 has a thickness of from 0.1 mm or more to 5 mm or less.

In one or more embodiments, the brace 14 has a length of 80 mm. In one or more embodiments, the brace 14 has a length of from 50 mm or more to 100 mm or less. In one or more embodiments, the brace 14 has a length of from 10 mm or more to 300 mm.

In one or more embodiments, the opposing sides 16, 18 have a length of 40 mm. In one or more embodiments, the opposing sides 16, 18 have a length of from 25 mm or more to 50 mm or less. In one or more embodiments, the opposing sides 16, 18 have has a length of from 5 mm or more to 100 mm or less.

In one or more embodiments, the tabs 26, 28 have a length of 5 mm. In one or more embodiments, the tabs 26, 28 have a length of from 2 mm or more to 8 mm or less. In one or more embodiments, the tabs 26, 28 have has a length of from 0 mm or more to 20 mm or less.

Shape memory support 12 includes a plurality of bracing ribs 24 extending therefrom. The bracing ribs 24 are designed in a generally semi-oval, semi-elliptical, or bulbous shape such that they match the inside of the incision where the retractor is to be placed. This shape is one feature that allows retractor 10 to hold an incision open, such as retractor 10 holding open the walls of an atrium.

As described above, shape memory support 12 is compressible in order to be inserted into an opening. The bracing ribs 24 move with the shape memory support 12 such that the overall area covered by the bracing ribs 24 also reduces, permitting the insertion of the bracing ribs 24 into an opening or incision. Then, when shape memory support 12 is permitted to decompress, the bracing ribs 24 move with the decompression of the shape memory support 12 to engage the inner walls or edges of the incision or inner walls to which the incision provides access. This contraction and subsequent expansion of the area covered by the bracing ribs 24 (and subsequent force applied to the inside of the opening) is another feature that allows retractor 10 to provide support for an opening, while holding itself in place.

In one or more embodiments, the area covered by the bracing ribs 24 and shape memory support 12 when uncompressed is larger than that of the opening or incision where the retractor 10 is to be inserted. This area can be designed to be larger than the opening such that when the shape memory support 12 is compressed, the subsequent decompression provides a greater exertion on the inside of the opening. As discussed below, a greater exertion can also be designed into the retractor 10 based on the materials that are chosen to make the retractor 10.

It should be appreciated that the bracing ribs 24 can be designed to have any shape or curve in order to fit a prescribed opening and, if desired, any inner walls to which the opening provides access. In one or more embodiments, bracing ribs 24 are nearly straight. In one or more embodiments, bracing ribs 24 have a significant curved or semi-elliptical shape. In one or more embodiments, all bracing ribs 24 all have a similar shape. In one or more embodiments, bracing ribs 24 are designed to have differing shapes, based on where each bracing rib 24 is located. In one or more embodiments, bracing ribs 24 have a coating in order to increase the grip that they provide to the inside of an opening. In one or more embodiments, bracing ribs 24 are designed to fit inside a cone-opening, as is performed in some atrial incisions.

As seen in FIG. 10, in one or more embodiments of the present invention, the bracing ribs 24 include apertures 36 therein. In one or more embodiments, the bracing ribs include apertures 36 at the end of the bracing ribs 24. In embodiments where apertures 36 are present, the apertures 36 offer a location for sutures such that the retractor 10 can be sutured into place for additional support.

In one or more embodiments, the bracing ribs 24 extend from the shape memory support 12 such that the bracing ribs are substantially parallel with each other. In one or more embodiments, bracing ribs 24 extend in differing directions in order to match the inside of an opening where retractor 10 is to be placed.

In one or more embodiments, bracing ribs 24 are coupled to shape memory support 12 by welding bracing ribs 24 thereto. In one or more embodiments, bracing ribs 24 are coupled to shape memory support 12 by applying an adhesive between bracing ribs 24 and shape memory support 12. In one or more embodiments, retractor 10 is molded or formed as one combined shape.

In one or more embodiments, retractor 10 can be formed using 3D printing, or additive manufacturing, in order to form the retractor as one combined shape. Examples of 3D printing include 3D polymer printing and 3D metal printing. 3D printing is a process of making a three-dimensional solid object of virtually any shape from a digital model. It is achieved using an additive process, where successive layers of material are laid down in different shapes.

In one or more embodiments, bracing ribs 24 are coupled to shape memory support 12 by a reversible or irreversible socket. In one or more embodiments, bracing ribs 24 are coupled to shape memory support 12 by a lock and key.

In one or more embodiments, a retractor includes twelve or more bracing ribs. In one or more embodiments, a retractor includes from eight or more to sixteen or less bracing ribs. In one or more embodiments, a retractor includes fifteen or less bracing ribs.

In one or more embodiments, the bracing ribs 24 have a length of 35 mm. In one or more embodiments, the bracing ribs 24 have a length of from 20 mm or more to 45 mm or less. In one or more embodiments, the bracing ribs 24 have a length of from 10 mm or more to 100 mm or less.

In one or more embodiments, the bracing ribs 24 have a thickness of 1.5 mm. In one or more embodiments, the bracing ribs 24 have a thickness of from 0.5 mm or more to 7 mm or less.

In one or more embodiments, the bracing ribs 24 have a curvature angle of from 20° or more to 50° or less with a radius of from 5 mm to 20 mm or less. In one or more embodiments, the bracing ribs 24 have a curvature angle of from 5° or more to 80° or less with a radius of from 2 mm or more to 80 mm or less.

As discussed above, retractor 10 is self-supporting once it is inserted into an opening. Once shape memory support 12 is compressed, placed inside an opening, and subsequently allowed to decompress, retractor 10 is able to support itself inside the opening. This is an improvement over known retractors that require an arm support to hold the retractor open.

This ability of retractor 10 is at least partly based on the flexibility and shape memory of the materials. This flexibility and shape memory allows retractor 10 to conform to different openings having different volumetric dimensions. Without being limited to any theory of operability, shape memory support 12 and bracing ribs 24 are able to exert force on the inside of the opening, thereby anchoring themselves on the inner walls of the opening, such as a left atrium. Therefore, any form of external support is rendered unnecessary.

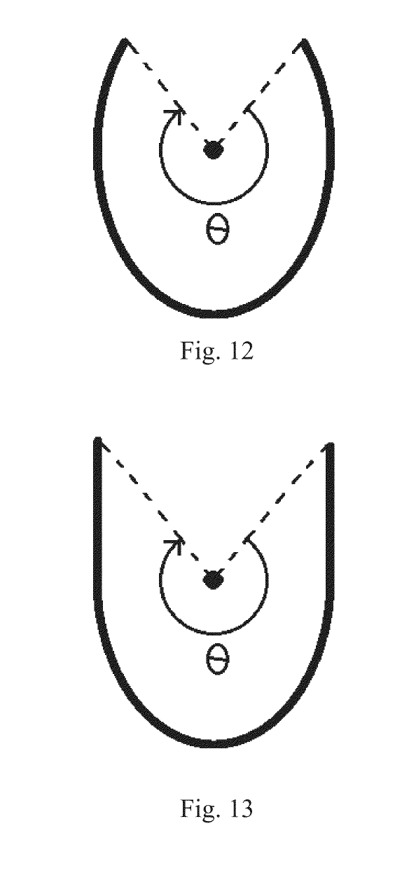

Either the brace 14 or the brace 14 and opposed sides 16, 18 of the shape memory support 12 can be said to have a radial angle θ. Since the invention is not limited to strictly semi-elliptical or semi-circular shape memory supports 12, the radial angle θ as shown in the figures is merely representative and is unable to be strictly defined. FIGS. 11 through 13 are representative showings of particular radial angles θ. The radial angle θ represents the angle formed by connecting the distal ends 20, 22 with an origin location. The origin location can be the center location of the full shape that would be formed if the shape memory support 12 were extended to a full shape.

In one or more embodiments, the radial angle θ is 90 degrees or more, such that it provides support for 90 degrees or more of an opening. Said another way, in one or more embodiments, the shape memory support 12 extends through 90 degrees or more.

In one or more embodiments, the radial angle θ is 320 degrees, or approximate thereto, such that it provides support for 320 degrees of an opening, or approximate thereto. Said another way, in one or more embodiments, the shape memory support 12 extends through 320 degrees, or approximate thereto.

In one or more embodiments, the radial angle θ is 120 degrees or more, in other embodiments 180 degrees or more, in other embodiments 240 degrees or more, in other embodiments 270 degrees or more, in other embodiments 300 degrees or more, and in other embodiments 320 degrees or more.

In one or more embodiments, the retractor 10 provides support for 180 degrees or more of an opening. In one or more embodiments, the retractor 10 provides support for 240 degrees or more of an opening. In one or more embodiments, the retractor 10 provides support for 320 degrees or more of an opening.

In one or more embodiments, the radial angle θ is 350 degrees or less. In one or more embodiments, the radial angle θ is 320 degrees or less. In one or more embodiments, the radial angle θ is 270 degrees or less. In one or more embodiments, the radial angle θ is 240 degrees or less.

With the understanding that a closed structure of the shape memory support 12, i.e., wherein the distal ends 20, 22 of the opposed sides 16, 18 touch is to be considered as having a periphery extending through 2π radians, in one or more embodiments, the periphery of the shape memory support 12 extends through at least π/2 radians. In other embodiments, the periphery of the shape memory support extends through at least 2π/3 radians, in other embodiments, at least 5π/6 radians, in other embodiments, at least π radians, in other embodiments, at least 7π/6 radians, in other embodiments, at least 4π/3 radians, in other embodiments at least 3π/2 radians, in other embodiments, at least 5π/3 radians, and in other embodiments, at least 7π/4 radians and in other embodiments, at least 16π/9 radians.

In one or more embodiments, retractor 10 can be designed such that the self supporting aspect is correlated for use in a left atrium. In these embodiments, the left atrium can be treated as an ellipsoid volume, such that its three axes are considered to be the major axis, minor axis, and diameter that echocardiographs measure in the apical four chamber view and parasternal long axis views, respectively.

The materials used to make retractor 10 must offer sufficient strength in order to allow retractor 10 to properly support an opening. These materials must also have sufficient flexibility such that retractor 10 can be compressed in order to be placed into the opening. Retractor should also be made from a material such that the insertion into a surgical opening does not have a detrimental effect on the surgical patient. Such material can be said to be an inert material.

In one or more embodiments, a retractor is made out of spring steel. In one or more embodiments, a retractor is made out of tempered spring steel. In one or more embodiments, a retractor is made out of a polymer. Suitable polymers are those polymers that have sufficient strength required to support the inside walls of an opening. Examples of suitable polymers include polycarbonate and polyurethane. In one or more embodiments, a retractor contains a coating, such as silicone, polybutadiene, rubber, carbon composite, or mixtures thereof In one or more embodiments, a coating is a biomedical coating.

Spring steel is a low-alloy, medium-to-high carbon, steel with a very high yield strength that gives it exceptional shape memory characteristics. Spring steel is able to be significantly deformed and easily decompress back into its original conformation. It has been previously used in applications that must stand high abuse such as car antennas, springs, piano wire, and lock-picks. It is generally easy to temper into a shape and can retain that shape for thousands of cycles.

Spring steel at all grades is already designed to withstand a severe amount of strain, but it can be designed to meet particular properties based on a desired application for a retractor. This analysis generally involves obtaining stress-strain curves at a variety of temperatures, lateral stress testing, and defective stress testing. After gathering this information, a material can be selected based on a particular application.

For embodiments where a retractor is made out of spring steel, it should be appreciated that there are many different grades of spring steel, and that optimal properties can be developed based on the desired application for a retractor. In one or more embodiments, the yield strength of a material used to make retractor 10 is from 400 MPa or more to 1100 MPa or less. In one or more embodiments, the elastic modulus of a material used to make retractor 10 is 185 GPa or more.

The materials that are used for making retractor 10 should be selected as to be reliable for many cycles (e.g. thousands of cycles) and have sufficient malleability. The materials should also be selected as to keep shape memory support 12 attached to bracing ribs 24.

As generally described above, retractor 10 is used by compressing or pinching shut shape memory support 12. Retractor 10 is then placed inside an opening, such as a left atrium. Once inserted into the opening, shape memory support 12 decompresses or self-expands and exerts a force onto the inside of the opening and the bracing ribs 24 can engage the inner walls, such as the inner walls of the heart. This exerted force and engagement of the inner walls allow retractor 10 to hold itself in place. The opening is then firmly supported for the remainder of an operation.

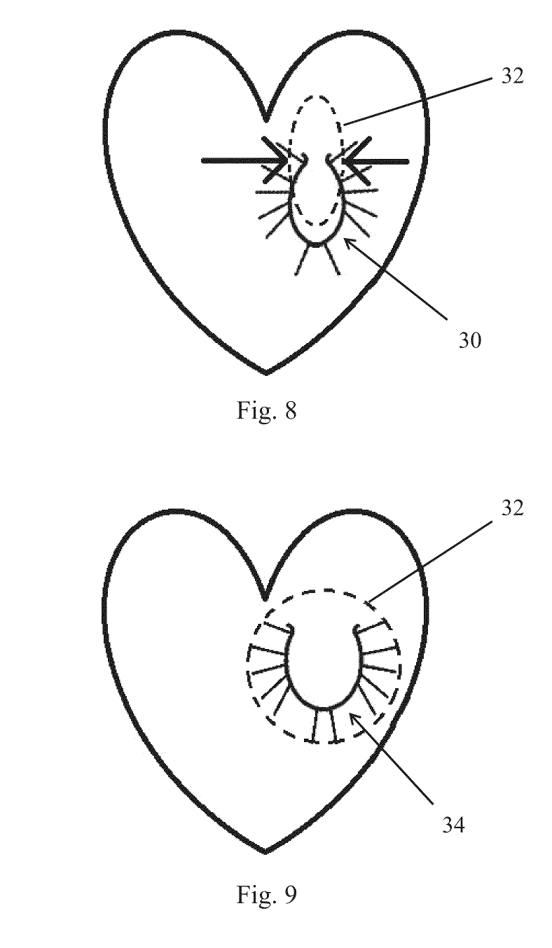

This method of use is represented schematically by FIGS. 7, 8, and 9. FIG. 7 shows arrows generally representing a means of manipulation of the retractor 10. The arrows also generally represent the direction of the manipulation or compression such that the retractor 10 takes a new shape as the manipulated retractor 30. In FIG. 8, it is shown that manipulated retractor 30 is then inserted into an opening 32. After insertion, and after the manipulation is removed, manipulated retractor 30 retains its rest shape, or approximate thereto, and becomes unmanipulated retractor 34 that holds open the opening 32 via bracing ribs 24 engaging the inner walls.

In embodiments where a retractor 10 includes bracing ribs 24 having apertures 36 therein, a method of using the retractor 10 can include the step of suturing the retractor 10 in a desired location using apertures 36.

The present invention offers one or more improvements over the known art. For one, the retractor increases the ease of access and field of vision for the surgeon and removes the need for handheld devices or devices that hinge off a sternal retractor, thus increasing the maneuverability of the surgical team.

Also, in one or more embodiments, the retractor is disposable. The retractor can also be pre-sterilized such that it can be immediately used with a surgery. In one or more embodiments, a retractor is packaged by itself for use in a surgery. In one or more embodiments, a retractor is packaged with other devices that are used for a surgery. In embodiments where the retractor is disposable, cross contamination is prevented. This further reduces the risk of infection. Materials chosen, and cost of producing, can be adjusted based on whether it is desired for the retractor to be disposable.

The present invention also offers reduced complexity and costs, particularly in view of the known art. The retractor eliminates the time spent on multiple adjustments of a traditional valve retractor, is free from attachment to a sternal retractor, and allows for fewer medical devices within the treated area.

In one or more embodiments, a retractor also includes a light source. Such a light source is preferably without wires as to not interfere with the surgery area. Such a light source can be selected from the group consisting of a chemiluminescent material, an LED light with an associated battery, a fiber optic fixture, and combinations thereof In one or more embodiments, a light source provides light that lasts from 2 or more hours to 3 or less hours.

In a particular embodiment, a semi-elliptical shape formed by the retractor 10 has an overall major axis of 5 mm and an overall minor axis of 4.5 mm, the shape memory support 12 has an overall length of 160 mm, the shape memory support 12 has a thickness of 0.8 mm, the shape memory support 12 extends through 320 degrees, the brace 14 has a length of 80 mm, the opposing sides 16, 18 have a length of 40 mm, the tabs 26, 28 have a length of 5 mm, the bracing ribs 24 have a length of 35 mm, and the bracing ribs 24 have a thickness of 0.5 mm.

In a particular embodiment, a retractor is designed to be used for a mitral valve replacement surgery. In another particular embodiment, a retractor is designed to be used for tricuspid valve replacements.

The mitral valve is a bicuspid valve in the heart that connects the left atrium to the left ventricle. Its primary function is to control the flow of blood, and opens as a result of increased pressure from blood buildup in the left atrium. Mitral valve replacement usually occurs when the valve becomes too tight for blood to flow into the left ventricle (stenosis), or too loose, causing blood to leak back into the atrium.

For a mitral valve replacement, a full incision in the chest is made. When the heart is accessed and put on cardiopulmonary bypass, the left atrium is opened. A particular embodiment of the present invention involves placing the improved retractor into the left atrium.

While a retractor is in place, infected growth can be cauterized off. The rest of the valve is then removed using a cauterizing tool. A fibrous annulus is put in the open valve area and then sutured around. The fibrous annulus provides a framework for the valve replacement. The valve is then threaded through the sutures, and the sutures were tied and cut. The sutures were color coded to preserve threading order. After, the heart was re-activated and the body was closed.

In light of the foregoing, it should be appreciated that the present invention significantly advances the art by providing an improved medical device. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A retractor for medical procedures comprising:
a shape memory support including a brace portion connecting opposed sides and
a plurality of bracing ribs extending from the shape memory support at the opposed sides.

2. The retractor of claim 1, wherein the periphery of the shape memory support formed by the brace portion and the opposed sides extends through at least π/2 radians.

3. The retractor of claim 2, wherein the periphery of the shape memory support formed by the brace portion and the opposed sides extends through from π/2 radians to 16π/9 radians.

4. The retractor of claim 1, wherein the shape memory support is formed of a composition selected from the group consisting of spring steel, polycarbonate, and polyurethane.

5. The retractor of claim 4, wherein the shape memory support is coated with a coating selected from the group consisting of silicone, polybutadiene, rubber, and a carbon composite.

6. The retractor of claim 1, wherein the bracing ribs are formed of a composition selected from the group consisting of spring steel, polycarbonate, and polyurethane.

7. The retractor of claim 5, wherein the bracing ribs are coated with a coating selected from the group consisting of silicone, polybutadiene, rubber, and a carbon composite.

8. The retractor of claim 1, wherein the plurality of bracing ribs are substantially parallel.

9. The retractor of claim 1, wherein the shape memory support has a length of from 100 mm to 200 mm.

10. The retractor of claim 1, wherein the shape memory support forms a semi-elliptical shape having an overall major axis of from 3 mm to 7 mm and an overall minor axis of from 2.5 mm to 6 mm.

11. The retractor of claim 1, wherein the bracing ribs have a length of from 20 mm to 45 MM.

12. The retractor of claim 1, wherein the opposed sides each provide a distal end having a tab extending therefrom.

13. The retractor of claim 1, wherein the bracing ribs include an aperture for inserting a suture.

14. The retractor of claim 13, wherein the aperture is located near an end of the bracing ribs.

15. The retractor of claim 1, wherein the retractor further comprises a light source.

16. A method of using a retractor for holding open an opening comprising the steps of:
providing a retractor, wherein the retractor comprises
a shape memory support including a brace portion connecting opposed sides and
a plurality of bracing ribs extending from the shape memory support at the opposed sides,
forming a surgical opening,
pinching the opposed sides of the shape memory support in an inward direction,
inserting the pinched retractor of said step of pinching into the surgical opening, and thereafter
allowing the opposed sides of the shape memory position to substantially revert to their original positions.

17. The method of claim 16, wherein the periphery of the shape memory support formed by the brace portion and the opposed sides extends through at least π/2 radians.

18. The method of claim 16, wherein the retractor is inserted into a left atrium.

19. The method of claim 16, wherein the opposed sides each provide a distal end having a tab extending therefrom, and the method further comprises the step of engaging the tabs with a medical tool in order to pinch the opposed sides of the shape memory support in an inward direction.

20. The method of claim 16, wherein the bracing ribs include an aperture for inserting a suture, and the method further comprises the step of suturing the retractor in a desired location.
